(19) United States
(12) Patent Application Publication
(10) Pub. No.: US 2026/0364205 A1
(43) Pub. Date: Nov. 19, 2026

(54) CHEMICAL MODIFICATION OF GENE EDITING SYSTEM COMPONENTS FOR ENHANCED INTRACELLULAR DELIVERY

(71) Applicant: BOARD OF TRUSTEES OF THE UNIVERSITY OF ARKANSAS, Little Rock, AR (US)

(72) Inventors: Christopher Nelson, Fayetteville, AR (US); Shilpi Agrawal, Fayetteville,, AR (US)

(73) Assignee: BOARD OF TRUSTEES OF THE UNIVERSITY OF ARKANSAS, Little Rock, AR (US)

(21) Appl. No.: 19/676,360

(22) Filed: May 13, 2026

Related U.S. Application Data
(60) Provisional application No. 63/805,007, filed on May 13, 2025.

(51) Int. Cl. A61K 48/00; C12N 9/22; C12N 15/11

(52) U.S. Cl. CPC A61K 48/005; C12N 9/226; C12N 15/111; C07K 2319/03; C12N 2310/20

(57) ABSTRACT

The invention relates to gene editing systems that include a modified component, where the modified component is associated with at least one compound, and where the compound is operable to enhance the cell permeability of the modified component. The invention also relates to methods of editing a gene of a cell by associating the cell with such gene editing systems. The method may occur in vitro or in vivo in a subject. The method may also be utilized to treat or prevent a disease or condition in the subject.

### CROSS-REFERENCE TO RELATED APPLICATIONS

This application claims priority to U.S. Provisional Patent Application No. 63/805,007, filed on May 13, 2025. The entirety of the aforementioned application is incorporated herein by reference.

### STATEMENT REGARDING FEDERALLY SPONSORED RESEARCH

This invention was made with government support under HT9425-24-1-0881 awarded by the Department of Defense. The government has certain rights in the invention.

### SEQUENCE DISCLOSURE STATEMENT

Pursuant to 37 C.F.R. § 1.834, Applicant has submitted a sequence listing in XML format (“Sequence Listing”). The name of the file containing the Sequence Listing is “AF42186.P071US.xml”. The date of the creation of the Sequence Listing is May 13, 2026. The size of the Sequence Listing is 4,000 bytes. Applicant hereby incorporates by reference the material in the Sequence Listing.

### BACKGROUND

A need exists for gene editing systems with enhanced cell permeability. Numerous embodiments of the present disclosure aim to address the aforementioned need.

### SUMMARY

In some embodiments, the present disclosure pertains to a gene editing system that includes a modified component. In some embodiments, the modified component is associated with at least one compound operable to enhance the cell permeability of the modified component. In some embodiments, the compound is associated with the modified component through a reversible bond.

Additional embodiments of the present disclosure pertain to methods of editing a gene of a cell by associating the cell with a gene editing system of the present disclosure. In some embodiments, the method occurs in vitro. In some embodiments, the method occurs in vivo in a subject by administering the gene editing system to the subject. In some embodiments, the method is utilized to treat or prevent a disease or condition in the subject.

### BRIEF DESCRIPTION OF THE DRAWINGS

A better understanding of the present invention can be obtained when the following detailed description is considered in conjunction with the following drawings, in which:

FIG. 1 summarizes solvent-accessible surface area (SASA) values highlighting key residues V743, S204, R1078, and L551 in Cas 9, which are highly exposed and amenable to modification for thiol-maleimide.

FIG. 2 shows a Python-based molecule (PyMOL)-generated image displaying the top four SASA residues. The SASA residues are as follows: S204, L551, V743, and R1078. These residues are identified as prime candidates for conjugation in Cas 9 modification. The structure is based on the crystal structure of Streptococcus pyogenes Cas 9 in complex with guide RNA and target DNA duplex (PDB ID: 4CMP).

FIG. 3 provides a protein sequence of Cas 9 highlighting key residues S204, L551, V743, and R1078 (SEQ ID NO: 1). These residues are targeted for substitution to cysteine (C) to facilitate thiol-maleimide conjugation for Cas 9 modification (SEQ ID NO: 1).

FIG. 4 provides an evaluation of wt-SpCas 9-His protein expression profile of SpCas 9-His using Ni-Sepharose affinity chromatography. The lanes are as follows: lane 1: protein ladder; lane 2—fraction from the wash step; lane 3—first 5 mL of the fraction from the elution step.

FIG. 5 shows a protein purification profile of C-terminal Cys-SpCas 9-His using Ni-Sepharose affinity chromatography. The lanes are as follows: lanes 1, 2—fraction from the wash step; lane 3—100 mM Imd fraction; lane 4—remaining 100 mM Imd fraction; lane 5—protein ladder; lane 6—250 mM Imd fraction.

FIG. 6 shows a Western blot analysis of SpCas 9-His expression. The lanes are as follows: lane 1: wt-SpCas 9-His protein; lane 2: Cys-modified SpCas 9-His; lane 3: V743C Cas 9 mutant; lane 4: S204C Cas 9 mutant. The pre-stained protein molecular weight marker shows the Cas 9 band at 160 kDa.

FIG. 7 shows an in vitro digestion of Cas 9. The lanes are as follows: L: Ladder, 1: DNA only; 2: Commercial Cas 9+DNA+gRNA; 3: SpCas 9+DNA+gRNA; 4: CysCas 9+DNA+gRNA; 5: V743C Cas 9+DNA+gRNA, 6: S204C Cas 9+DNA+gRNA. The sgRNA:Cas 9:DNA ratio is 20:20:1.

FIG. 8 provides a summary of Tracking of Indels by Decomposition (TIDE) and Inference of CRISPR Edits (ICE) analysis. The results indicate that the Cas 9 mutants display robust editing efficiency across five CysCas 9 mutations.

FIG. 9 demonstrates that TIDE and ICE analysis displays on-target editing across five CysCas 9 variants relative to untreated control groups and Cas 9 delivered with a scrambled guide RNA.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Over the past decade, gene editing systems, such as the clustered regularly interspaced short palindromic repeats (CRISPR)/Cas nuclease (Cas) (CRISPR/Cas) systems, have revolutionized gene editing by providing highly specific and programmable methods for introducing changes into the genome. However, one of the significant challenges in therapeutic applications has been the efficient delivery of the Cas protein (e.g., Cas 9 proteins) to cells, especially in the context of systemic administration for conditions such as Duchenne Muscular Dystrophy (DMD).

Adeno-associated virus (AAV) has been widely used as a delivery vehicle for CRISPR/Cas systems. However, AAV is associated with several limitations, including immunogenicity, on-target mutagenesis, and the inability to re-dose due to immune responses against the viral capsid. Additionally, non-viral delivery methods have so far shown limited efficiency, particularly when targeting hard-to-reach tissues such as muscle.

As such, a need exists for gene editing systems with enhanced cell permeability. Numerous embodiments of the present disclosure aim to address the aforementioned need.

In some embodiments, the present disclosure pertains to a gene editing system that includes a modified component. In some embodiments, the modified component is associated with at least one compound operable to enhance the cell permeability of the modified component.

Additional embodiments of the present disclosure pertain to methods of editing a gene of a cell by associating the cell with a gene editing system of the present disclosure. In some embodiments, the method occurs in vitro. In some embodiments, the method occurs in vivo in a subject by administering the gene editing system to the subject. In some embodiments, the method is utilized to treat or prevent a disease or condition in the subject.

As set forth in more detail herein, the gene editing systems and methods of the present disclosure can have numerous embodiments. Additionally, the gene editing systems and methods of the present disclosure can have numerous advantages and applications.

### Gene Editing Systems

The systems and methods of the present disclosure may utilize various gene editing systems. For instance, in some embodiments, the gene editing system includes, without limitation, epigenetic modifiers, base editors, transcriptional regulators, recombinases, nucleases, clustered regularly interspaced short palindromic repeats (CRISPR)/Cas nuclease (Cas) (CRISPR/Cas) systems, or combinations thereof.

In some embodiments, the gene editing systems of the present disclosure include one or more guide RNAs configured to direct the gene editing system to a target nucleic acid sequence. In some embodiments, the gene editing system components may be wild type or engineered variants exhibiting altered specificity, activity, or stability.

The gene editing systems of the present disclosure may exhibit various effects. For instance, in some embodiments, the gene editing systems of the present disclosure enable gene disruption, insertion, deletion, base conversion, transcriptional modulation, epigenetic modification, or combinations of such effects.

### Modified Components

The gene editing systems of the present disclosure may include and be associated with various modified components. For instance, in some embodiments, the modified component includes a protein or enzyme of the gene editing system. In some embodiments, the protein or enzyme is engineered to improve stability, activity, specificity, or delivery.

In some embodiments, the modified component includes a fusion protein. In some embodiments, the fusion protein includes a gene editing protein linked to one or more functional domains, such as cell-penetrating peptides (CPPs), targeting ligands, nuclear localization signals, endosomal escape domains, or combinations thereof.

In some embodiments, the modified component includes chemically modified nucleic acids. In some embodiments, the chemically modified nucleic acids include modified guide RNAs with enhanced stability or targeting efficiency.

In some embodiments, the modified component is conjugated via covalent or non-covalent interactions. In some embodiments, the modified component may be associated with delivery systems, such as lipid nanoparticles, polymers, viral vectors.

### Compounds

The modified components of the gene editing systems of the present disclosure may be associated with various compounds. For instance, in some embodiments, the compound includes a cell-penetrating peptide (CPP). In some embodiments, the CPP is operable to facilitate transport of the modified component across a cell membrane. In some embodiments, the cell-penetrating peptide includes, without limitation, TAT protein-derived peptides (TAT), model amphipathic peptides (MAP), polyarginine-based peptides, poly-lysine-based peptides, Pep-1, VE-cadherin-derived peptides (pVEC), or combinations thereof. In some embodiments, the CPP includes between 5 and about 30 amino acids. In some embodiments, the CPP includes cationic and/or amphipathic residues that promote membrane translocation.

In some embodiments, the CPP is covalently or non-covalently associated with the modified component, including via linker chemistries such as maleimide-thiol coupling or other conjugation strategies. In some embodiments, the CPP facilitates intracellular delivery of proteins, ribonucleoprotein (RNP) complexes, or nucleic acids, including guide RNAs.

In some embodiments, the CPP is further modified to include targeting ligands, endosomal escape domains, or tissue-specific delivery moieties to enhance delivery efficiency and specificity. In some embodiments, multiple CPPs or combinations of CPPs with delivery vehicles are employed to improve cellular uptake and functional gene editing efficiency.

Association of Compound with Modified Component

The modified components of the gene editing systems of the present disclosure may be associated with compounds in various manners. For instance, in some embodiments, the modified component is associated with a single compound. In some embodiments, the modified component is associated with a plurality of the same compounds. In some embodiments, the modified component is associated with a plurality of different compounds. In some embodiments, the modified component retains gene editing activity while associated with the compound.

In some embodiments, the compounds of the present disclosure are associated with the modified components of the present disclosure through a reversible bond. In some embodiments, the reversible bond is cleavable in a reducing intracellular environment to release the modified component from the compound.

In some embodiments, the compounds of the present disclosure are associated with the modified components of the present disclosure at a location that is internal to the modified component and not at a terminal end of the modified component. In some embodiments, a plurality of compounds are associated with a modified component at two or more distinct regions of the modified component. In some embodiments, the two or more distinct regions include spatially separated internal regions of a modified component.

In some embodiments, the modified components of the present disclosure include a protein. In some embodiments, the compounds of the present disclosure are associated with the protein at one or more internal amino acid residues.

In some embodiments, the compounds of the present disclosure are associated with the modified components of the present disclosure through a disulfide bond. In some embodiments, the compounds of the present disclosure are associated with the modified components of the present disclosure through a thiol-maleimide linkage.

In some embodiments, the compounds of the present disclosure are associated with at least one cysteine residue of the modified components of the present disclosure. In some embodiments, the cysteine residue is introduced to the modified component by substitution of a residue with cysteine. In some embodiments, the cysteine substitution provides a thiol group for conjugation of the compound to the modified component. In some embodiments, the substitution is performed by site-directed mutagenesis. In some embodiments, the residue is a solvent-accessible residue. In some embodiments, the solvent-accessible residue is identified by solvent-accessible surface area (SASA) analysis.

### CRISPR/Cas Systems

In some embodiments, the gene editing systems of the present disclosure include a CRISPR/Cas system. In some embodiments, the CRISPR/Cas system includes at least one Cas nuclease and at least one guide RNA. In some of such embodiments, the modified component of the CRISPR/Cas system includes the Cas nuclease. In some embodiments, the Cas nuclease retains nuclease activity while associated with the compound and complexed with a guide RNA.

In some embodiments, the compounds of the present disclosure are associated with at least one cysteine residue of a Cas nuclease. In some embodiments, the compound is associated with one or more internal cysteine residues of the Cas nuclease. In some embodiments, the cysteine residue is introduced to the Cas nuclease by substitution of a residue with cysteine. In some embodiments, the Cas nuclease includes, without limitation, class 2 of Cas nucleases, Cas 9, Streptococcus pyogenes Cas 9 (SpCas 9), Staphylococcus aureus Cas 9 (SaCas 9), Cas12a, Cas Φ, CasΦ2, Cpf1, or combinations thereof.

In some embodiments, the Cas nuclease includes Cas 9. In some embodiments, the Cas 9 includes Streptococcus pyogenes Cas 9 (SpCas 9). In some embodiments, Cas 9 has an amino acid sequence that includes SEQ ID NO: 1. In some embodiments, Cas 9 has an amino acid sequence with at least 65% sequence identity to SEQ ID NO: 1. In some embodiments, Cas 9 has an amino acid sequence with at least 70% sequence identity to SEQ ID NO: 1. In some embodiments, Cas 9 has an amino acid sequence with at least 75% sequence identity to SEQ ID NO: 1. In some embodiments, Cas 9 has an amino acid sequence with at least 80% sequence identity to SEQ ID NO: 1. In some embodiments, Cas 9 has an amino acid sequence with at least 85% sequence identity to SEQ ID NO: 1. In some embodiments, Cas 9 has an amino acid sequence with at least 90% sequence identity to SEQ ID NO: 1. In some embodiments, Cas 9 has an amino acid sequence with at least 95% sequence identity to SEQ ID NO: 1. In some embodiments, Cas 9 has an amino acid sequence with at least 99% sequence identity to SEQ ID NO: 1. In some embodiments, the compound is associated with at least one cysteine residue of the Cas 9. In some embodiments, the cysteine residue is introduced to the Cas 9 by substitution of a residue with cysteine. In some embodiments, the Cas 9 includes at least one cysteine substitution. In some embodiments, the cysteine substitution includes, without limitation, S204C, L291C, L551C, V743C, K755C, R1078C, or combinations thereof.

### Gene Delivery Kits

In some embodiments, the gene editing systems of the present disclosure are part of a gene delivery kit. In some embodiments, the gene delivery kit is a non-viral gene delivery kit. In some embodiments, the gene delivery kit includes the modified component and instructions for associating the modified component with a cell.

Association of Gene Editing Systems with Cells

The gene editing systems of the present disclosure may be associated with cells in various manners. For instance, in some embodiments, cell association occurs in vitro. In some embodiments, in vitro cell association occurs in a petri dish.

In some embodiments, cell association occurs in vivo in a subject. In some of such embodiments, cell association includes administering the gene editing systems of the present disclosure to the subject. In some embodiments, the administration occurs by a method that includes, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof.

### Diseases or Conditions

In some embodiments, the gene editing systems and methods of the present disclosure are utilized to treat, prevent, or ameliorate one or more diseases or conditions in a subject. In some embodiments, the disease or condition is a genetic disorder, including those arising from mutations, deletions, insertions, or dysregulated gene expression.

In some embodiments, the disease or condition includes a neuromuscular disorder. In some embodiments, the neuromuscular disorder includes muscular dystrophy, including but not limited to Duchenne muscular dystrophy (DMD), Becker muscular dystrophy, limb-girdle muscular dystrophy, facioscapulohumeral muscular dystrophy, or congenital muscular dystrophies.

In some embodiments, the gene editing systems and methods of the present disclosure are configured to restore, correct, or modulate expression of a target gene associated with the disease. For instance, in some embodiments relating to DMD, the gene editing systems and methods of the present disclosure are configured to modify the dystrophin gene (DMD gene), including through exon skipping, exon deletion, exon insertion, or correction of mutations to restore functional dystrophin expression.

In some embodiments, the gene editing systems and methods of the present disclosure are delivered to muscle tissue, including skeletal muscle, cardiac muscle, or smooth muscle. In some embodiments, delivery may occur via systemic or localized administration.

In some embodiments, the gene editing systems and methods of the present disclosure may be utilized to improve muscle function, reduce degeneration, or slow disease progression. In some embodiments, the gene editing systems and methods of the present disclosure may be used in combination with other therapeutic agents.

### Subjects

The gene editing systems and methods of the present disclosure may be utilized to treat or prevent a disease or condition in various subjects. For instance, in some embodiments, the subject is a mammal (e.g., a human). In some embodiments, the subject is a human being. In some embodiments, the subject is a domesticated animal. For instance, in some embodiments, the subject is a dog or a cat. In some embodiments, the subject is a cow, a horse, a non-human primate, a mouse, a rat, or a rabbit.

### Applications and Advantages

Compared to current viral delivery systems, the gene editing systems and methods of the present disclosure offer several advantages. For instance, the gene editing systems and methods of the present disclosure reduce immunogenicity, thereby allowing for repeated dosing without triggering an immune response. Additionally, the gene editing systems and methods of the present disclosure eliminate the risks associated with viral vector integration into the host genome, which can cause unwanted mutations.

Additionally, the gene editing systems and methods of the present disclosure are faster, safer, and more cost-effective. For instance, in some embodiments, the gene editing systems and methods of the present disclosure advance the field of gene editing by offering a safer and more efficient alternative to AAV-based delivery systems. The gene editing systems and methods of the present disclosure also address key challenges associated with viral delivery, including immunogenicity and limited tissue specificity, while also improving the potential for repeated dosing without immune clearance.

As such, the gene editing systems and methods of the present disclosure can be utilized for various applications. For instance, in some embodiments, the gene editing systems and methods of the present disclosure offer a new pathway for treating genetic diseases that require precise gene editing, such as Duchenne Muscular Dystrophy (DMD).

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. CRISPR-Cas 9 Systems with Enhanced Delivery to Cells

This Example pertains to the improvement of the delivery efficiency of CRISPR-Cas 9 gene editing systems to target tissues. One approach to enhance the delivery of CRISPR-Cas 9 involves the use of cell-penetrating peptides (CPPs), which facilitate the transport of macromolecules across the cell membrane. The integration of CPPs into Cas 9 using cysteine-disulfide chemistry offers a promising solution, allowing for the reversible attachment of CPPs to Cas 9, thereby improving its cellular uptake while preserving its biological activity. The use of thiol-maleimide conjugation chemistry enables a reversible modification that can facilitate intracellular release of Cas 9 once inside the target cells, thus addressing delivery barriers while minimizing off-target effects and immune responses.

This Example builds on the recent advances in Cas 9 protein engineering and nanoparticle delivery to create a self-delivering gene-editing tool that could overcome the limitations of current viral and non-viral delivery systems. In particular, this Example describes a novel method for enhancing the delivery of Cas 9, the key component of CRISPR-based gene editing, by conjugating it with cell-penetrating peptides (CPPs) using thiol-maleimide chemistry (e.g., cysteine-disulfide chemistry). The conjugation significantly improves Cas 9's ability to cross cell membranes and enter target tissues, particularly skeletal muscle, making it a highly effective tool for gene therapy applications. The reversible nature of the chemical bond ensures that Cas 9 is released once inside the cell, retaining its full gene-editing functionality.

By conjugating CPPs to specific surface exposed cysteine residues on Cas 9, the Cas 9 protein can be actively transported across cell membranes, improving its cellular uptake and nuclear localization. Once inside the cell, the disulfide bond is cleaved in the reducing environment of the cytoplasm, releasing the Cas 9 to perform its genome-editing function. This chemical modification strategy could be applied to other proteins, broadening its utility in delivering various therapeutic proteins, including epigenetic modifiers and base editors.

A key aspect of this Example involves identifying solvent-accessible residues on the Cas protein that are highly exposed and suitable for chemical modification, followed by substitution of these residues with cysteine (C) for further conjugation. This method can be broadly applied to different Cas proteins, depending on the requirements of the specific gene editing application.

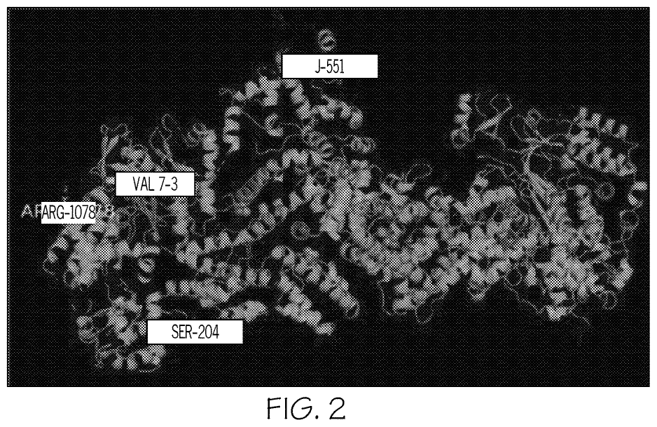

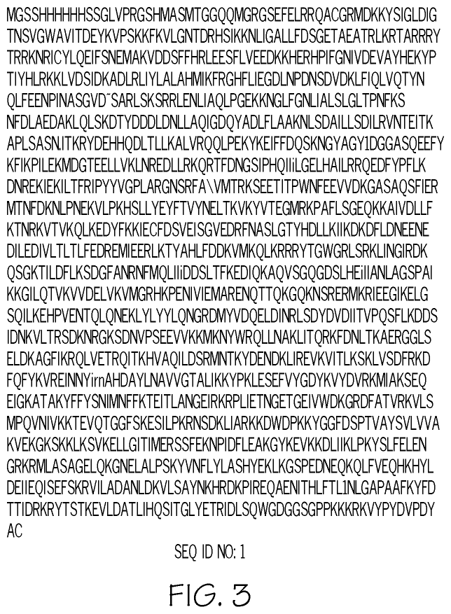

The Cas 9 protein was analyzed for solvent-accessible surface area (SASA), which highlighted several key residues: V743, S204, R1078, and L551. These residues were identified as highly exposed and ideal for chemical modification (FIG. 2). Using PyMOL, a molecular visualization tool, Applicant generated a structural model of SpCas 9 to visualize these residues in 3D space and confirm their accessibility (FIG. 2). Based on this analysis, these amino acids were selected for substitution with cysteine (C), as this allows for the formation of a disulfide bond through thiol-maleimide chemistry (FIG. 3).

In practice, the selected residues (S204, L551, V743, and R1078) are substituted with cysteine through site-directed mutagenesis. This creates reactive thiol groups at the selected positions, enabling the conjugation of CPPs via disulfide bonds. The thiolmaleimide reaction is reversible within the reducing environment inside cells, allowing the CPPs to dissociate from Cas 9 once it enters the cytoplasm, releasing Cas 9 in its active form for genome editing.

The Example provides several advantages over existing Cas 9 delivery methods, particularly when compared to viral delivery systems such as AAV. For instance, the addition of CPPs significantly enhances the cellular uptake of Cas 9, thereby allowing it to bypass membrane barriers more effectively. Additionally, the thiol-maleimide chemistry ensures that the modification is reversible, allowing for the CPPs to detach once Cas 9 reaches its target, thus reducing potential interference with Cas 9's gene-editing function. Moreover, the modularity of this approach allows for different CPPs to be conjugated based on the target tissue or cell type, thereby providing flexibility in therapeutic applications. Furthermore, unlike viral delivery systems, this approach minimizes the immune response, allowing for repeated dosing without eliciting a strong immune reaction.

Several key experiments were conducted to demonstrate the effectiveness of the modifying Cas 9 with cell-penetrating peptides (CPPs) using thiol-maleimide chemistry—each providing evidence that this approach enhances Cas 9 delivery and functionality.

### Example 1.1. Structural Integrity of Cas 9 Cysteine Mutants

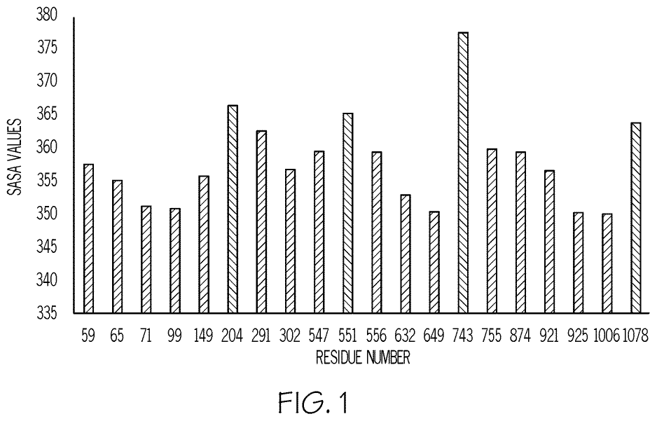

To confirm that the cysteine substitutions did not negatively affect the structural integrity of the Cas 9 protein, Applicant utilized solvent-accessible surface area (SASA) analysis, followed by structural visualization using PyMOL. This analysis identified key residues (S204, L551, V743, and R1078) that were highly exposed and optimal for chemical conjugation (FIGS. 1 and 2). The PyMOL-generated images (FIG. 2) and protein sequence analysis (FIG. 3) confirmed that these modifications were made at strategic points on the protein, maintaining its overall structure while allowing for functional enhancement through conjugation.

### Example 1.2. Protein Modification and Purification

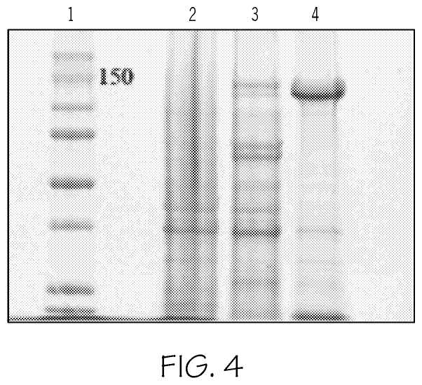

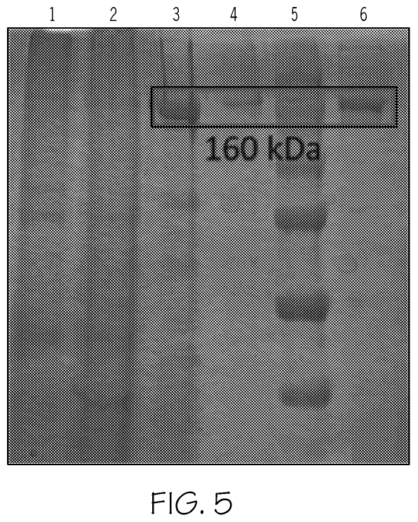

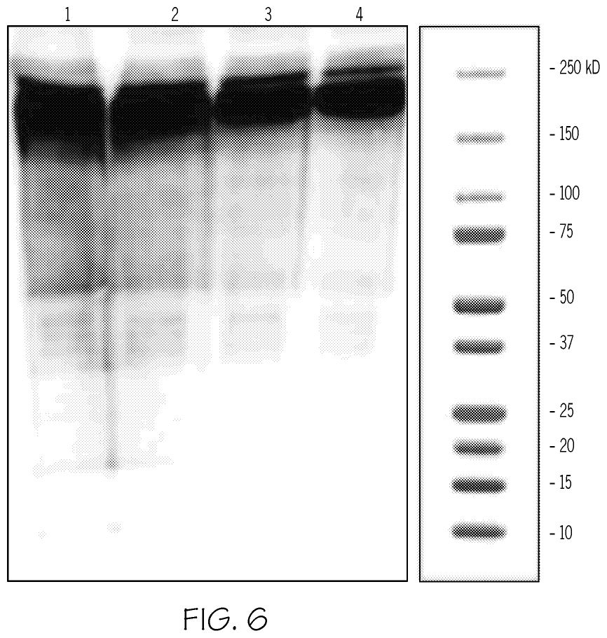

The initial step involved the substitution of solvent-accessible residues (S204, L551, V743, and R1078) on the SpCas 9 protein with cysteine (C) to allow for thiol-maleimide conjugation. Site-directed mutagenesis was employed to introduce cysteine at these specific positions, followed by expression and purification of both wild-type and modified SpCas 9-His proteins. Using Ni-Sepharose affinity chromatography, Applicant successfully purified the wild-type SpCas 9-His (FIG. 4) and the cysteine-modified Cas 9 variants (FIG. 5). This confirmed that the modification process did not interfere with the protein's overall structure or ability to be purified in high yields. The presence of clear, distinct bands on the Western blot analysis at the expected 160 kDa mark (FIG. 6) confirmed the successful expression and correct molecular weight of the Cas 9 proteins.

### Example 1.3. Functional Validation with In Vitro Cleavage Assays

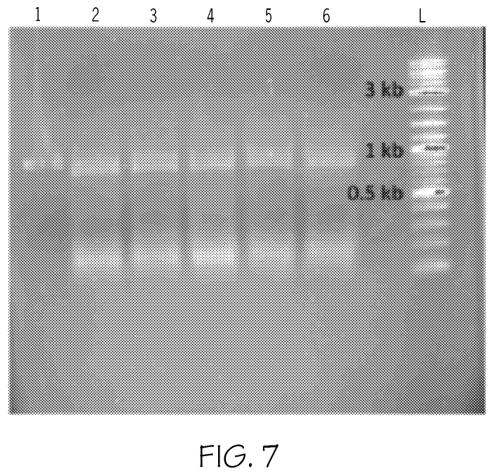

Next, Applicant performed in vitro DNA cleavage assays to test the functionality of the modified Cas 9 proteins. These assays used plasmid DNA as the target, with a 60:60:1 (nM) ratio of guide RNA (gRNA), SpCas 9, and plasmid DNA. The results demonstrated that only the SpCas 9-gRNA complexes (both wild-type and cysteine-modified variants) were able to digest the plasmid DNA, indicating that the modified Cas 9 retained its DNA-cleaving ability post-modification (FIG. 7). Importantly, the cleavage efficiency of the modified Cas 9 was comparable to that of commercial Cas 9, demonstrating that the modification did not impair Cas 9's gene-editing function.

### Example 1.4. Genome Editing Efficiency Analysis of the Cas 9 Mutants (TIDE and ICE)

Genome editing efficiency of engineered Cas 9 variants were evaluated using Sanger sequencing followed by TIDE (Tracking of Indels by Decomposition) and ICE (Inference of CRISPR Edits) analysis. These methods enable quantitative assessment of insertion/deletion (indel) frequencies at the target locus, providing a robust measure of CRISPR-Cas 9 activity.

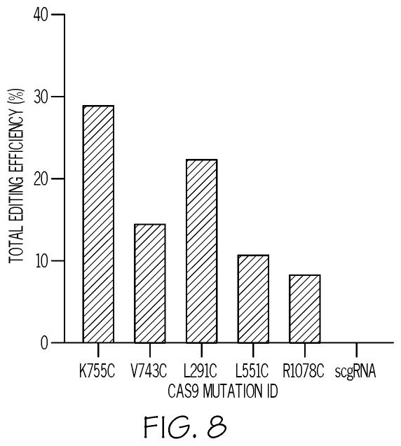

TIDE analysis revealed that multiple cysteine-engineered Cas 9 variants (i.e., K755C, V743C, L291C, L551C, and R1078C) retain functional genome editing capability, with measurable indel formation at the target site (FIG. 8). Among the variants tested, K755C exhibited the highest editing efficiency (~28-30%), followed by L291C (~20-22%) and V743C (~14-15%). Additional variants, including L551C and R1078C, demonstrated moderate but detectable editing activity (~8-12%), indicating partial retention of catalytic function. The non-targeting control (scgRNA) showed negligible editing, confirming assay specificity.

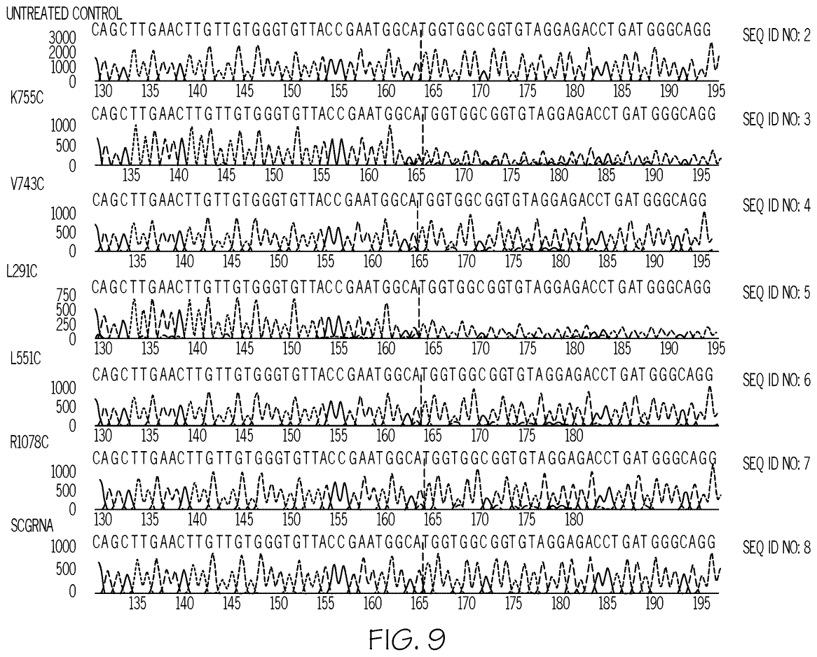

These findings were further supported by ICE analysis of Sanger sequencing chromatograms, which showed characteristic signal deconvolution downstream of the predicted Cas 9 cut site (FIG. 9). Variants with higher editing efficiencies (e.g., K755C and L291C) displayed pronounced mixed sequencing peaks consistent with heterogeneous indel populations, whereas lower-activity variants showed reduced but still detectable signal disruption. Control samples exhibited clean, unperturbed sequencing traces, confirming the absence of editing.

Collectively, TIDE and ICE analyses demonstrate that site-specific cysteine substitutions at solvent-exposed residues of Cas 9 can preserve genome editing activity while enabling chemical conjugation. Importantly, the variability in editing efficiency across variants provides insight into structurally permissive regions for engineering, supporting rational selection of Cas 9 constructs for downstream functionalization, including conjugation to cell-penetrating peptides.

### Example 1.5. Summary

As part of the proof of concept, Applicant compared the performance of the cysteine-modified Cas 9 with commercially available Cas 9. The results of the Western blot analysis and in vitro cleavage assays demonstrated that the modified Cas 9 not only maintained its gene-editing functionality but also exhibited enhanced delivery potential due to its ability to conjugate with CPPs. This suggests that the disclosure can improve cellular uptake while retaining Cas 9's core gene-editing capabilities, making it suitable for therapeutic applications.

In sum, this Example offers a novel, efficient, and flexible approach to delivering Cas 9 into target cells, thereby addressing key limitations of current delivery methods. By modifying solvent-accessible residues with CPPs using cysteine-disulfide chemistry, the present disclosure provides a robust solution for enhancing gene editing efficiency while minimizing potential side effects. With the data and experimental validation presented, the present disclosure holds great promise for advancing the field of gene therapy.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A gene editing system comprising a modified component, wherein the modified component is associated with at least one compound operable to enhance the cell permeability of the modified component, and wherein the compound is associated with the modified component through a reversible bond.

2. The gene editing system of claim 1, wherein the gene editing system is selected from the group consisting of epigenetic modifiers, base editors, transcriptional regulators, recombinases, nucleases, clustered regularly interspaced short palindromic repeats (CRISPR)/Cas nuclease (Cas) (CRISPR/Cas) systems, or combinations thereof.

3. The gene editing system of claim 1, wherein the compound comprises a cell-penetrating peptide, wherein the cell-penetrating peptide is operable to facilitate transport of the modified component across a cell membrane.

4. The gene editing system of claim 1, wherein the reversible bond is cleavable in a reducing intracellular environment to release the modified component from the compound.

5. The gene editing system of claim 1, wherein the compound is associated with at least one cysteine residue of the modified component.

6. The gene editing system of claim 5, wherein the cysteine residue is introduced to the modified component by substitution of a residue with cysteine.

7. The gene editing system of claim 1, wherein the compound is associated with the modified component through a disulfide bond.

8. The gene editing system of claim 1, wherein the compound is associated with the modified component at a location that is internal to the modified component and not at a terminal end of the modified component.

9. The gene editing system of claim 1, wherein a plurality of compounds are associated with the modified component at two or more distinct regions of the modified component, and wherein the two or more distinct regions comprise spatially separated internal regions of the modified component.

10. The gene editing system of claim 1, wherein the modified component comprises a protein, and wherein the compound is associated with the protein at one or more internal amino acid residues.

11. The gene editing system of claim 1, wherein the gene editing system comprises a clustered regularly interspaced short palindromic repeats (CRISPR)/Cas nuclease (Cas) (CRISPR/Cas) system, wherein the CRISPR/Cas system comprises at least one Cas nuclease and at least one guide RNA, and wherein the modified component comprises the Cas nuclease.

12. The gene editing system of claim 11, wherein the compound is associated with one or more internal cysteine residues of the Cas nuclease.

13. The gene editing system of claim 12, wherein the cysteine residue is introduced to the Cas nuclease by substitution of a residue with cysteine.

14. The gene editing system of claim 11, wherein the Cas nuclease is selected from the group consisting of class 2 of Cas nucleases, Cas 9, Streptococcus pyogenes Cas 9 (SpCas 9), Staphylococcus aureus Cas 9 (SaCas 9), Cas12a, Cas Φ, CasΦ2, Cpf1, or combinations thereof.

15. The gene editing system of claim 11, wherein the Cas nuclease comprises Cas 9, wherein the Cas 9 has an amino acid sequence comprising SEQ ID NO: 1 or a sequence with at least 65% sequence identity to SEQ ID NO: 1, wherein the compound is associated with at least one cysteine residue of the Cas 9, and wherein the cysteine residue is introduced to the Cas 9 by substitution of a residue with cysteine.

16. The gene editing system of claim 15, wherein the Cas 9 comprises at least one cysteine substitution selected from the group consisting of S204C, L291C, L551C, V743C, K755C, R1078C, or combinations thereof.

17. A method of editing a gene of a cell, said method comprising:
associating the cell with a gene editing system, wherein the gene editing system comprises a modified component, wherein the modified component is associated with at least one compound operable to enhance the cell permeability of the modified component, and wherein the compound is associated with the modified component through a reversible bond.

18. The method of claim 17, wherein the method occurs in vitro.

19. The method of claim 17, wherein the method occurs in vivo in a subject, and wherein the method comprises administering the gene editing system to the subject.

20. The method of claim 19, wherein the method is utilized to treat or prevent a disease or condition in the subject.

21. The method of claim 20, wherein the disease or condition comprises a neuromuscular disorder, wherein the neuromuscular disorder is selected from the group consisting of muscular dystrophy, Duchenne muscular dystrophy (DMD), Becker muscular dystrophy, limb-girdle muscular dystrophy, facioscapulohumeral muscular dystrophy, congenital muscular dystrophies, or combinations thereof.

22. The method of claim 19, wherein the subject is a human being.

23. The method of claim 17, wherein the compound comprises a cell-penetrating peptide, wherein the cell-penetrating peptide facilitates transport of the modified component across a cell membrane.

24. The method of claim 17, wherein the reversible bond is cleavable in a reducing intracellular environment to release the modified component from the compound.

25. The method of claim 17, wherein the compound is associated with the modified component at a location that is internal to the modified component and not at a terminal end of the modified component.

26. The method of claim 17, wherein a plurality of compounds are associated with the modified component at two or more distinct regions of the modified component, and wherein the two or more distinct regions comprise spatially separated internal regions of the modified component.

27. The method of claim 17, wherein the modified component comprises a protein, and wherein the compound is associated with the protein at one or more internal amino acid residues.

28. The method of claim 17, wherein the compound is associated with at least one cysteine residue of the modified component.

29. The method of claim 17, wherein the gene editing system comprises a clustered regularly interspaced short palindromic repeats (CRISPR)/Cas nuclease (Cas) (CRISPR/Cas) system, wherein the CRISPR/Cas system comprises at least one Cas nuclease and at least one guide RNA, and wherein the modified component comprises the Cas nuclease.

30. The method of claim 29, wherein the compound is associated with one or more internal cysteine residues of the Cas nuclease.

31. The method of claim 29, wherein the Cas nuclease comprises Cas 9, wherein the Cas 9 has an amino acid sequence comprising SEQ ID NO: 1 or a sequence with at least 65% sequence identity to SEQ ID NO: 1, wherein the compound is associated with at least one cysteine residue of the Cas 9, and wherein the cysteine residue is introduced to the Cas 9 by substitution of a residue with cysteine.

32. The method of claim 31, wherein the Cas 9 comprises at least one cysteine substitution selected from the group consisting of S204C, L291C, L551C, V743C, K755C, R1078C, or combinations thereof.


SEQUENCE LISTING (1 sequence)

SEQ ID NO: 1 - protein, 1435 aa, unidentified
MGSSHHHHHHSSGLVPRGSHMASMTGGQQMGRGSEFELRRQACGRMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGPPKKKRKVYPYDVPDYAC